(19) United States
(12) Patent Application Publication
(10) Pub. No.: US 2010/0303936 A1
(43) Pub. Date: Dec. 2, 2010

(54) METHOD OF REDUCING FAT ACCUMULATION AND INDUCING WEIGHT LOSS

(75) Inventor: Isaac Cohen, Piedmont, CA (US)

Correspondence Address: WILSON, SONSINI, GOODRICH & ROSATI, 650 PAGE MILL ROAD, PALO ALTO, CA, 94304-1050, US

(73) Assignee: Bionovo, Inc. A Delaware Corporation

(21) Appl. No.: 12/769,054

(22) Filed: Apr. 28, 2010

Related U.S. Application Data
(60) Provisional application No. 61/309,341, filed on Mar. 1, 2010.
(60) Provisional application No. 61/173,553, filed on Apr. 28, 2009.

(51) Int. Cl. A61K 36/488; A61P 3/04; A61K 36/296

(52) U.S. Cl. 424/757; 424/725

(57) ABSTRACT

Administration of estrogenic extracts having ERα-agonistic activity results in reduced fat accumulation, induced weight loss, or both.

This application claims priority under 35 U.S.C. §119(e) to U.S. provisional applications 61/309,341, filed Mar. 1, 2010, and 61/173,553, filed Apr. 28, 2009, each of which is incorporated herein by reference in its entirety.

### BACKGROUND OF THE INVENTION

The rates of obesity and overweight have increased drastically over recent decades, and the prevalence of obesity and overweight tends to increase with age. Obesity and overweight are correlated with numerous pathologies, including sleep apnea, cardiovascular disease, diabetes, and general deterioration of patients' feelings of wellness. Aside from self-image issues relating to obesity and overweight, which can be considerable, overweight and obese patients can suffer from multiple symptoms aside from the aforementioned pathologies, including fatigue, drowsiness, lack of motivation, anhedonia, and depression. Inflammatory diseases, such as arthritis, have been shown to be exacerbated by excess weight, which may be an effect of increased adipocyte proliferation and the concomitant increased activity of adipose tissue specific cytokines known as adipokines.

Obesity, overweight, and their related pathologies and symptomatologies are serious problems in the industrialized world, where the balance of calories burned and calories consumed can often tip in the direction of consumption. Although it is generally recommended that obesity and overweight be addressed first by a regimen of reduced caloric intake and increased exercise, this is not always practicable (e.g. with the old and infirm), and is often ineffective. In addition to age in general, obesity and weight gain are independently correlated with menopause, as are other correlated pathologies, such as depression, sleep loss, fatigue, drowsiness and anhedonia. In fact, menopause is associated with about a 10 to 15 lb. weight gain and a redistribution of fat to the abdomen. Distribution of fat increases in the trunk (p<0.001) and decreases in the legs (p<0.05) for postmenopausal women. This maldistribution of fat increases the risk for metabolic syndrome, cardiovascular disease and type 2 diabetes.

Indeed, it is believed that changes during the menopausal transition, rather than the aging process itself, are related to changes in body weight and fat distribution associated with perimenopause and postmenopause. In age-matched women, a higher percentage of fat tissue is found in perimenopausal and postmenopausal women (p<0.001) compare to premenopausal and postmenopausal women, and women who are receiving hormone therapy (HT). The shift to a more android fat distribution in perimenopausal and postmenopausal women may be reversed by hormone replacement therapy (HRT). This distribution has been shown to be reversed by HRT.

Thus, obesity presents a challenge in need of a solution. Many products have been offered to the public without a prescription. The purveyors of such products often claim that the products will suppress appetite, increase energy levels and reduce weight; however the credibility of these claims is questionable. Clinical trials of much-awaited prescription weight-loss drugs have been less than promising, especially in light of the recent high profile failure lorcaserin to achieve a 5% separation in weight loss as compared to placebo, which cast some doubt on whether drug approval would be forthcoming. Of course this casts the claims of over-the-counter remedies in all that much greater doubt.

While hormone replacement therapy may reverse the distribution of fat to the abdomen in perimenopausal and postmenopausal women, HRT is not without its risks. The estrogens employed in hormone replacement therapy are not tissue selective. They tend to activate estrogen receptor alpha (ERα) adipose tissue as well as breast and uterine tissue. In adipose tissue, estrogen tends to reverse distribution of fat to the abdomen, which is a desirable effect. However, in breast and uterine tissue estrogen can have a growth-stimulating effect, which places the patient at a greater risk for breast and uterine cancer.

Given the growing incidence of obesity and overweight among the aging population, and the potential benefits of reducing or maintaining weight within acceptable levels, there is thus a need for a product that will reduce the accumulation of fat, especially in aging patients, and more particularly in menopausal women. There is further a need for a product that will induce weight loss, especially in aging patients, and more particularly in menopausal women. Further there is a need for compositions and methods for reducing body fat accumulation and inducing weight loss that do not induce ERα response in breast and uterine tissue.

### SUMMARY OF THE INVENTION

The inventor has found that certain extracts of herbs activate estrogen receptor alpha (ERα) in adipose tissue but not in breast or uterine tissue. In vitro extracts of Radix Anamarrhena, Radix Glycyrrhiza, Radix Pueraria, Herba Epimedii agonize ERα-mediated gene transcription. However, in vivo, extracts of Radix Glycyrrhiza and Radix Pueraria produced ERα-mediated reduction of fat accumulation in ovariectomized mice fed a high fat diet, but did not cause tissue proliferation in mammary and uterine tissues in the same animal model, thus indicating that the extracts do not agonize ERα in mammary and uterine tissues. This is in contrast to estradiol (E2), which induced uterine tissue weight gain and elongation and branching of ducts in mammary tissue in the mouse model. Two particular extracts, of Radix Glycyrrhiza and Radix Pueraria, activated ERα in vitro, but did not cause proliferation of MCF7 cells in vitro. These extracts were tested in the animal model of ovariectomized high fat diet mice, and demonstrated potential for reducing fat accumulation, and especially android (abdominal) fat accumulation, and inducing weight loss, in perimenopausal and postmenopausal women.

Thus, some embodiments, the invention provides a method of reducing fat accumulation, inducing weight loss, or both, comprising administering to a subject an estrogen receptor alpha (ERα) agonizing amount of a plant extract comprising one or more members selected from the group consisting of extracts of Radix Anamarrhena, Radix Glycyrrhiza, Radix Pueraria, Herba Epimedii, and combinations thereof. In some embodiments, the body fat accumulation that is reduced is abdominal body fat accumulation. In particular embodiments, the method comprises administering to a subject an ERα-agonizing amount of a composition comprising an extract of Radix Glycyrrhiza (e.g. G. uralensis and/or G. glabra), Radix Pueraria (e.g. P. lobata), or both.

In some embodiments, the invention provides a composition for reducing fat accumulation, in particular abdominal body fat accumulation, inducing weight loss, or both, comprising an estrogen receptor alpha (ERα) agonizing amount of a plant extract comprising one or more members selected from the group consisting of extracts of Radix Anamarrhena, Radix Glycyrrhiza, Radix Pueraria, Herba Epimedii, and combinations thereof. In particular embodiments, the compositions comprises an ERα-agonizing amount of an extract of Radix Glycyrrhiza (e.g. G. uralensis and/or G. glabra), Radix Pueraria (e.g. P. lobata), or both.

In some embodiments, the invention provides a method of reducing fat accumulation (especially abdominal fat accumulation) inducing weight loss, or both, comprising administering to a menopausal, perimenopausal or postmenopausal subject an estrogen receptor alpha (ERα) agonizing amount of a plant extract comprising one or more members selected from the group consisting of extracts of Radix Anamarrhena, Radix Glycyrrhiza, Radix Pueraria, Herba Epimedii, and combinations thereof. In particular embodiments, the plant extract comprises an ERα-agonizing amount of an extract of Radix Glycyrrhiza (e.g. G. uralensis and/or G. glabra), Radix Pueraria (e.g. P. lobata), or both.

Although the inventor believes that the compositions and methods described herein reduce fat accumulation, particularly abdominal fat accumulation, and induce weight loss, by agonizing (activating) the ERα estrogen receptor in adipose tissue, especially abdominal adipose tissue, while at the same time not agonizing (activating) ERα in mammary and uterine tissue, and while the inventor considers this to be a rational hypothesis based upon the in vitro and in vivo activities of the compositions according to the invention, he also recognizes that the biological pathway of operation of the compositions in vivo can be complex, and the observed abdominal fat reduction in ovariectomized mice may involve alternate biological pathways that are not described herein. It is not necessarily the intention of the inventor that the compositions and methods described herein be limited in all cases by the hypothetical biological pathway of selective (adipose-directed) ERαagonism. Thus, in some embodiments, the invention provides a composition for reducing fat accumulation (especially abdominal fat accumulation), inducing weight loss, or both, in a menopausal, perimenopausal or postmenopausal woman, comprising an amount of a plant extract comprising one or more members selected from the group consisting of extracts of Radix Anamarrhena, Radix Glycyrrhiza, Radix Pueraria, Herba Epimedii, and combinations thereof, wherein the amount of plant extract is sufficient to reduce fat accumulation (especially abdominal fat accumulation) and/or induce weight loss, especially in perimenopausal, menopausal or postmenopausal women. In some embodiments, the plant extract comprises fat-accumulation reducing amount of an extract of Radix Glycyrrhiza (e.g. G. uralensis and/or G. glabra), Radix Pueraria (e.g. P. lobata), or both. In some embodiments, the plant extract comprises an abdominal fat-accumulation reducing amount of an extract of Radix Glycyrrhiza (e.g. G. uralensis and/or G. glabra), Radix Pueraria (e.g. P. lobata), or both. In some embodiments, the plant extract comprises a weight loss inducing amount of an extract of Radix Glycyrrhiza (e.g. G. uralensis and/or G. glabra), Radix Pueraria (e.g. P. lobata), or both. As described herein, compositions comprising a combination of an extracts of Radix Glycyrrhiza and Radix Pueraria, or an extract of a combination comprising Radix Glycyrrhiza and Radix Pueraria, are in some cases preferred.

Also, in some embodiments, the invention provides a method for reducing fat accumulation (especially abdominal fat accumulation) inducing weight loss, or both, in a menopausal, perimenopausal or postmenopausal women, comprising administering to such a woman an amount of a plant extract comprising one or more members selected from the group consisting of extracts of Radix Anamarrhena, Radix Glycyrrhiza, Radix Pueraria, Herba Epimedii, and combinations thereof, wherein the amount of plant extract is sufficient to reduce fat accumulation (especially abdominal fat accumulation) and/or induce weight loss. In particular embodiments, the plant extract that is administered to the women comprises a fat-accumulation reducing amount of an extract of Radix Glycyrrhiza (e.g. G. uralensis and/or G. glabra), Radix Pueraria (e.g. P. lobata), or both. In some embodiments, the plant extract that is administered comprises an abdominal fat-accumulation reducing amount of an extract of Radix Glycyrrhiza (e.g. G. uralensis and/or G. glabra), Radix Pueraria (e.g. P. lobata), or both. In some embodiments, the plant extract that is administered comprises a weight loss inducing amount of an extract of Radix Glycyrrhiza (e.g. G. uralensis and/or G. glabra), Radix Pueraria (e.g. P. lobata), or both. As described herein, compositions comprising a combination of an extracts of Radix Glycyrrhiza and Radix Pueraria, or an extract of a combination comprising Radix Glycyrrhiza and Radix Pueraria, are in some cases preferred.

Also, in some embodiments, the invention provides a method for reducing fat accumulation (especially abdominal fat accumulation) inducing weight loss, or both, in a mammal, such as a human, comprising administering to such a mammal, such as a human, an amount of a plant extract comprising one or more members selected from the group consisting of extracts of Radix Anamarrhena, Radix Glycyrrhiza, Radix Pueraria, Herba Epimedii, and combinations thereof, wherein the amount of plant extract is sufficient to reduce fat accumulation (especially abdominal fat accumulation) and/or induce weight loss. In particular embodiments, the plant extract that is administered to the mammal, such as a human, comprises a fat-accumulation reducing amount of an extract of Radix Glycyrrhiza (e.g. G. uralensis and/or G. glabra), Radix Pueraria (e.g. P. lobata), or both. In some embodiments, the plant extract that is administered comprises an abdominal fat-accumulation reducing amount of an extract of Radix Glycyrrhiza (e.g. G. uralensis and/or G. glabra), Radix Pueraria (e.g. P. lobata), or both. In some embodiments, the plant extract that is administered comprises a weight loss inducing amount of an extract of Radix Glycyrrhiza (e.g. G. uralensis and/or G. glabra), Radix Pueraria (e.g. P. lobata), or both. As described herein, compositions comprising a combination of an extracts of Radix Glycyrrhiza and Radix Pueraria, or an extract of a combination comprising Radix Glycyrrhiza and Radix Pueraria, are in some cases preferred.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the results of an experiment in which ovariectomized mice were fed a high fat diet (HFD) and treated with vehicle (control), estradiol (E2), an extract of Glycyrrhiza (3) or an extract of Pueraria (39). Ovariectomized female mice fed an HFD and treated with vehicle (control) continued to gain weight, whereas mice treated with E2, 3 or 39 experienced weight loss during the same 42 day treatment period.

FIG. 2 shows the mean weight in grams of abdominal fat recovered after 42 days of treatment from vehicle-treated (control), E2-, 3- and 39-treated ovariectomized mice fed an HFD. Each of E2, 3, and 39 reduced abdominal fat accumulation during the 42 day treatment period as compared to the control.

FIG. 3 shows the mean weight in grams of uteri recovered after 42 days of treatment from vehicle-treated (control), E2-, 3- and 39-treated ovariectomized mice fed an HFD. Only E2 induced significant weight gain in the uterus. Extracts 3 and 39 did not mimic E2's effect on the uterus.

FIG. 4 shows the mean weight in grams of mammary gland recovered from mice after 42 days of treatment from vehicle-treated (control), E2-, 3-, and 39-treated ovariectomized mice fed an HFD. Each of E2, 3 and 39 induced lower mammary gland weight as compared to the control. It was observed by histology, however, that E2, in addition to reducing mammary gland weight, also induced elongation and branching of mammary ducts, whereas 3 and 39 had no such effects on the mammary glands.

FIG. 5 shows a comparison of up- and down-regulation of genes in abdominal fat, as obtained in a gene expression profile obtained by a microarray study. As can be seen, each of E2, 3 and 39 up-regulated and down-regulated a relatively large number of genes in the microarray study.

FIG. 6 shows a comparison of up- and down-regulation of genes in uteri obtained from mice that were treated with E2, 3 and 39. As can be seen, E2 induced and repressed numerous genes in the uteri, whereas 3 and 39 had little effect on gene transcription in the uteri.

FIG. 7 shows a comparison of up- and down-regulation of genes in mammary glands obtained from mice treated with E2, 3 and 39. As can be seen, E2 induced and repressed numerous genes in the mammary glands, whereas 3 had practically not effect on gene transcription and 39 had little effect on gene transcription in the mammary glands.

### DETAILED DESCRIPTION OF THE INVENTION

The invention provides a method of reducing body fat (e.g. abdominal fat) accumulation, of inducing weight loss, or of both in animal subjects. In some embodiments, the subjects are human subjects, such as menopausal, perimenopausal and postmenopausal women. It has been found that administration of ERα agonistic plant extracts from particular plant species causes reversal of abdominal fat accumulation and induces weight loss in an animal model—i.e. ovariectomized mice fed a high fat diet (HFD). In embodiments of the invention, administration of a composition comprising an extract of Radix Anamarrhena, Radix Glycyrrhiza, Radix Pueraria, or Herba Epimedii to high-fat fed ovariectomized female mice reverses abdominal fat accumulation. In particular embodiments, administration of an extract of Radix Glycyrrhiza or Radix Pueraria induces weight loss in ovariectomized HFD mice. As the ovariectomized HFD mouse model is an accepted model for weight gain associated with menopause in humans, it follows that compositions of the invention containing an extract, or extracts, of Radix Anamarrhena, Radix Glycyrrhiza, Radix Pueraria, or Herba Epimedii, or combinations thereof, will reduce fat accumulation (especially abdominal fat accumulation) and/or induce weight loss in perimenopausal, menopausal and postmenopausal women. Moreover, compositions of the invention containing an extract, or extracts, of Radix Glycyrrhiza, Radix Pueraria, or combinations thereof, will reduce fat accumulation (especially abdominal fat accumulation) and/or induce weight loss in perimenopausal, menopausal and postmenopausal women. Thus, the compositions described herein may be used in methods of reducing fat accumulation (e.g. abdominal fat accumulation), inducing weight loss, or both, in perimenopausal, menopausal and/or postmenopausal women.

The inventor has found that compositions taught herein possess tissue-specific estrogen receptor alpha (ERα) agonistic activity in vivo. In particular, the compositions taught herein possess ERα agonistic activity in adipose tissue, but not in mammary gland and uterine tissue, in ovariectomized mice, which is an animal model of menopausal (including perimenopausal and postmenopausal) women. Thus, in some embodiments described herein, there is provided a method of selectively agonizing Estrogen Receptor alpha (ERα) in adipose tissue, comprising administering an effective amount of a plant extract comprising one or more members selected from the group consisting of extracts of Radix Anamarrhena, Radix Glycyrrhiza, Radix Pueraria, Herba Epimedii, and combinations thereof. At doses effective to reduce accumulation of fat and/or induce weight gain, compositions described herein fail to stimulate mammary gland proliferation or differentiation (e.g. duct elongation and branching) or uterine weight increase. In some embodiments, the plant extract comprises two or more members selected from the group consisting of extracts of Radix Anamarrhena, Radix Glycyrrhiza, Radix Pueraria, and Herba Epimedii. In some embodiments, the plant extract comprises three or more members selected from the group consisting of extracts of Radix Anamarrhena, Radix Glycyrrhiza, Radix Pueraria, and Herba Epimedii. In some embodiments, the plant extract comprises extracts of Radix Anamarrhena, Radix Glycyrrhiza, Radix Pueraria, and Herba Epimedii. In some embodiments, the method produces a reduction in abdominal fat accumulation. In some embodiments, the plant extract comprises extracts of Radix Glycyrrhiza, Radix Pueraria, or combinations thereof. In some specific embodiments, the plant extract contains an extract of Radix Glycyrrhiza as the sole active ingredient. In some embodiments, the plant extract contains an extract of Radix Pueraria as the sole active ingredient. In some embodiments, the composition contains a combination of Radix Glycyrrhiaz and Radix Pueraria extracts, or an extract of a combination of Radix Glycyrrhiza and Radix Pueraria, as the sole active ingredient. In some embodiments, the plant extract comprises a combination of extracts of Radix Glycyrrhiza and Radix Pueraria. In some embodiments, the plant extract comprises an ethanol:water extract of Radix Glycyrrhiza or Herba Epimedii. In some embodiments, the plant extract comprises an ethyl acetate partition of an extract of Radix Anamarrhena, Radix Glycyrrhiza, Radix Pueraria, or Herba Epimedii. In some embodiments, the plant extract comprises an aqueous extract of Herba Epimedii. In some embodiments, the plant extract comprises a mixture of two or more of: an ethanol:water extract of Radix Glycyrrhiza or Herba Epimedii, an ethyl acetate partition of an extract of Radix Anamarrhena, Radix Glycyrrhiza, Radix Pueraria, or Herba Epimedii, or an aqueous extract of Herba Epimedii. In some embodiments, the plant extract comprises one or both of an ethanol:water extract of Radix Glycyrrhiza and/or an ethyl acetate partition of an extract of Radix Pueraria. In some embodiments the plant extract comprises an ethanol:water extract of Radix Glycyrrhiza as the sole active ingredient. In some embodiments the plant extract comprises an ethyl acetate partition of an extract of Radix Pueraria as the sole active ingredient. In some embodiments, the plant extract comprises a combination of an ethanol:water partition of an extract of Radix Glycyrrhiza and an ethyl acetate partition of an extract of Radix Pueraria, and the combination is the sole active ingredient. In some embodiments, the composition comprises from about 1 mg to about 100 g (e.g. 10 mg to 50 g, 30 mg to 30 g) of an extract of Radix Glycyrrhiza (or an ethanol:water partition of an extract of Radix Glycyrrhiza) and about 1 mg to about 100 g (e.g. 10 mg to 50 g, 30 mg to 30 g) of an extract of Radix Pueraria (or an ethyl acetate partition of an extract of Radix Pueraria.

As used herein, the term “active ingredient” refers to an herbal extract or partition of an herbal extract (e.g. an ethanol:water or ethyl acetate partition of an herbal extract) that has tissue-selective ERα agonistic activity. In particular, an “active ingredient” is an ingredient that causes ERα-mediated phenotypic change in adipose tissue, but causes little or no ERα-mediated phenotypic change in uterine and mammary tissue.

The phrase “sole active ingredient” means that the recited ingredient (e.g. herbal extract, partition of an herbal extract, combination of herbal extracts, or combination of partitions of herbal extract or extracts) is the sole ingredient that possesses in vivo or in vitro biological activity. In some embodiments, the recited ingredient is the only ingredient in the composition that possesses ER-agonistic and/or ER-antagonistic effect. In some embodiments, the recited ingredient is the sole ingredient in the composition that possesses ERα agonistic effect. In some embodiments, the recited ingredient is the sole ingredient in the composition that possesses a beneficial medicinal effect.

### Herbs

As used herein, Glycyrrhiza refers to either or both of Glycyrrhiza uralensis and Glycyrrhiza glabra. In particular, the root or radix of Glycyrrhiza is used to prepare the extracts employed in the compositions described herein.

As used herein, Pueraria refers to Puraria lobata. In particular, the root or radix of Pueraria is used to prepare the extracts employed in the compositions described herein.

As used herein, Epimedii refers to Epimedium grandiflorum Morr. In particular, the

As used herein Anamarrhena refers to Anamarrhena asphodeloides Bge.

Aqueous extracts of various plants (PEs) were subjected to partitioning with various solvents. The table below sets forth partitions of water extracts of herbs that were tested for ERα agonist activity. In particular, 3 and 39 were found to have ERα agonist activity in vitro.

ERα-Agonist Activity-Possessing Extracts

| | | Fraction | | |
|---|---|---|---|---|
| | | Ethanol: | Ethyl | Aqueous |
| Name | | Water | acetate | (Post-Partition) |
| Radix Anamarrhena | ZM | | 11 | |
| Radix Glycyrrhiza | GC | 3 | 12 | |
| Radix Pueraria | GG | | 39 | |
| Herba Epimedi | YY | 28 | 37 | 28B |

### Pharmaceutical Compositions

Extracts of Radix Anamarrhena, Radix Glycyrrhiza, Radix Pueraria, Herba Epimedii, or combinations of two or more thereof, may be prepared as above in either solution or dried form. Extracts of extracts of Radix Anamarrhena, Radix Glycyrrhiza, Radix Pueraria, Herba Epimedii, or combinations of two or more thereof, may be used to prepare a pharmaceutical composition (medicament) for the treatment of one or more conditions or disease states treatable with an estrogenic composition. Such pharmaceutical compositions (medicaments) may optionally incorporate one or more pharmaceutically acceptable excipients. In a solution form, an extracts of Radix Anamarrhena, Radix Glycyrrhiza, Radix Pueraria, Herba Epimedii, or combinations of two or more thereof, may be administered in the form a flavored or unflavored tea. In some embodiments, some flavoring, e.g. sweetening, may be desirable to counteract the bitter flavor of the extract. Solutions can also be prepared from dried extract, in tea or elixir forms. Again, flavoring, such as sweetening may be desirable. Taste-masking may be employed to improve patient acceptance of the pharmaceutical composition.

A dried extract may be formulated as an orally-available form, such as in a capsule, tablet, caplet, etc. A capsule may be prepared by measuring a suitable amount of the dry extract into one or more gelatin capsule shells and assembling the capsule(s). Tablets and caplets may be prepared by combining the dry extract with one or more binders and optionally one or more disintegrants. Tablets, caplets, capsules, etc. may be coated, e.g. with an enteric coating, to prevent stomach upset.

Either a dried extract or a concentrated extract solution may be combined with one or more gelling agents and inserted into a gel capsule. Alternatively, a dried extract or concentrated extract solution may be combined with a gelling agent and optionally one or more flavoring agents for oral administration as an edible gel or a non-flavored variant may be administered as a rectal suppository gel or gel capsule.

A unit dose of extract may be characterized by an equivalent amount of dried extract contained within the dosage form. For example, in some embodiments, a unit dosage may contain 1 mg to about 10 g (10,000 mg) of dried extract, or the equivalent thereof. In some embodiments, the unit dose will contain about 1 mg to about 10 mg, about 1 mg to about 100 mg, about 1 mg to about 1000 mg (1 g), about 1 mg to about 10000 mg (10 g) of dried extract, or the equivalent thereof. In some embodiments, the unit dose contains about 10 mg to about 100 mg, about 10 mg to about 1000 mg or about 10 mg to about 10000 mg of dried extract or the equivalent thereof. In some embodiments, the unit dose contains about 100 mg to about 5000, about 100 mg to about 2500 mg, about 100 mg to about 2000 mg, about 100 mg to about 1500 mg, about 100 to about 1000, about 100 to about 800 mg of dried extract, or the equivalent thereof. In some embodiments, a daily dose comprises about 1 to about 100 grams dry weight of extract of Anemarrhena asphodeloides Bunge, which may be prepared in a single unit or in divided units, and may be given in a single dose or in two, three, four or more divided doses. In some embodiments, the daily dose is about 10 to about 100 grams, about 10 to about 80 grams, about 10 to about 60 grams, about 10 to about 40 grams, about 20 to about 100 grams, about 20 to about 80 grams, about 20 to about 60 grams, about 20 to about 40 grams dry weight of extract of Anemarrhena asphodeloides Bunge. In some embodiments, the daily dose is about 10, about 15, about 20, about 25, about 30, about 35, about 40, about 45, about 50, about 55, about 60, about 65, about 70, about 75, about 80, about 85, about 90, about 95, or about 100 grams dry weight of extract of Radix Anamarrhena, Radix Glycyrrhiza, Radix Pueraria, Herba Epimedii, or combinations of two or more thereof. An equivalent of a dried extract of Radix Anamarrhena, Radix Glycyrrhiza, Radix Pueraria, Herba Epimedii, or combinations of two or more thereof is an amount of a dry, liquid, gel or other mixture of Radix Anamarrhena, Radix Glycyrrhiza, Radix Pueraria, Herba Epimedii, or combinations of two or more thereof containing the same amount of apoptotic active as a dried extract of Radix Anamarrhena, Radix Glycyrrhiza, Radix Pueraria, Herba Epimedii, or combinations of two or more thereof. Thus, 30 mL of a tea containing 0.5 mg/mL of dried extract of Radix Anamarrhena, Radix Glycyrrhiza, Radix Pueraria, Herba Epimedii, or combinations of two or more thereof is a unit dose equivalent to 15 mg of dried Radix Anamarrhena, Radix Glycyrrhiza, Radix Pueraria, Herba Epimedii, or combinations of two or more thereof; and a tablet containing 100 mg each of dried extract of Radix Anamarrhena, Radix Glycyrrhiza, Radix Pueraria, Herba Epimedii, or combinations of two or more thereof, a binder, a filler, a disintegrant is equivalent to 100 mg of dried extract neat.

### Examples

The following illustrative examples further elucidate what the inventor considers to be his invention. The examples are not intended to be limiting, but to demonstrate enablement of the invention.

### Example 1

### Plant-Derived Estrogen Receptor Alpha (ERα) Agonists Selectively Reverse Abdominal Fat Accumulation Without Increasing Mammary Gland and Uterus Proliferation

In humans, menopause is associated with about a 10-15 pound weight gain and a redistribution of fat to the abdomen, which increases the risk for metabolic syndrome, cardiovascular disease and type 2 diabetes. Some previously-described estrogens, such as estradiol (E2) can decrease body fat mass, as well as intra-abdominal and intrapelvic fat, in postmenopausal women. Unfortunately, hormone therapy (HT) with estrogens such as E2 is associated with an increased risk of breast cancer and cardiovascular disease. Estrogens that retain their beneficial effect on fat accumulation, but do not promote cell proliferation and cancer, should have a profound impact on the long-term health and well-being of postmenopausal women.

While estrogen receptor-alpha (ERα) mediates the effects of estrogens on fat accumulation and mammary gland cell proliferation, the inventor hypothesized that some ERα agonists might exert tissue specific effects that make them safer than estrogens, such as estradiol (E2), which are generally used in hormone therapy (HT). To test this hypothesis, over 50 crude plant extracts (PEs) used in traditional Chinese medicine were screened for ERα activity. Two PEs had ERα activity using an ERE-luciferase reporter, like E2, but did not stimulate the proliferation of MCF-7 human breast cancer cells, unlike E2. These two PEs were identified by internal identification numbers 3 (an ethanol:water fraction obtained from a water extract of Glycyrrhiza) and 39 (an ethyl acetate fraction obtained from a water extract of Pueraria).

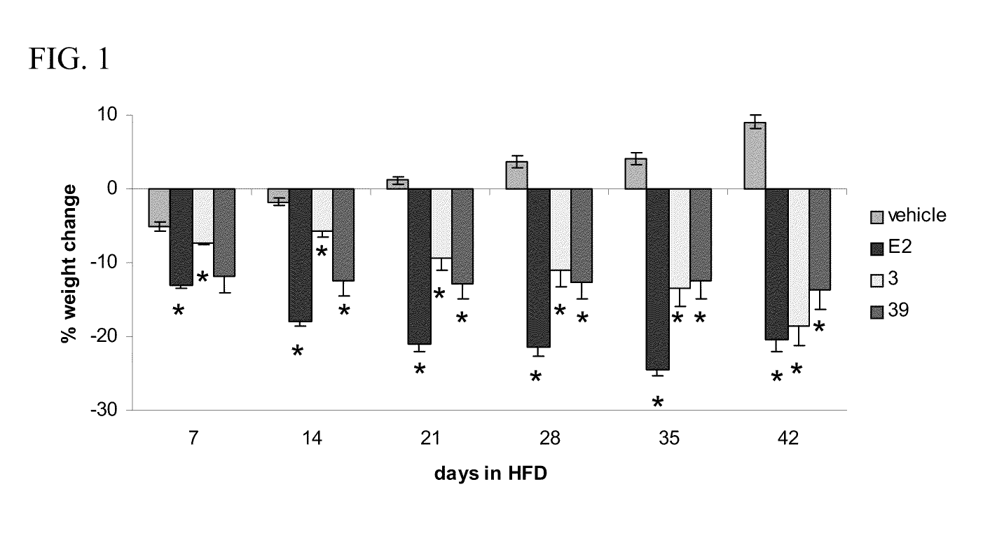

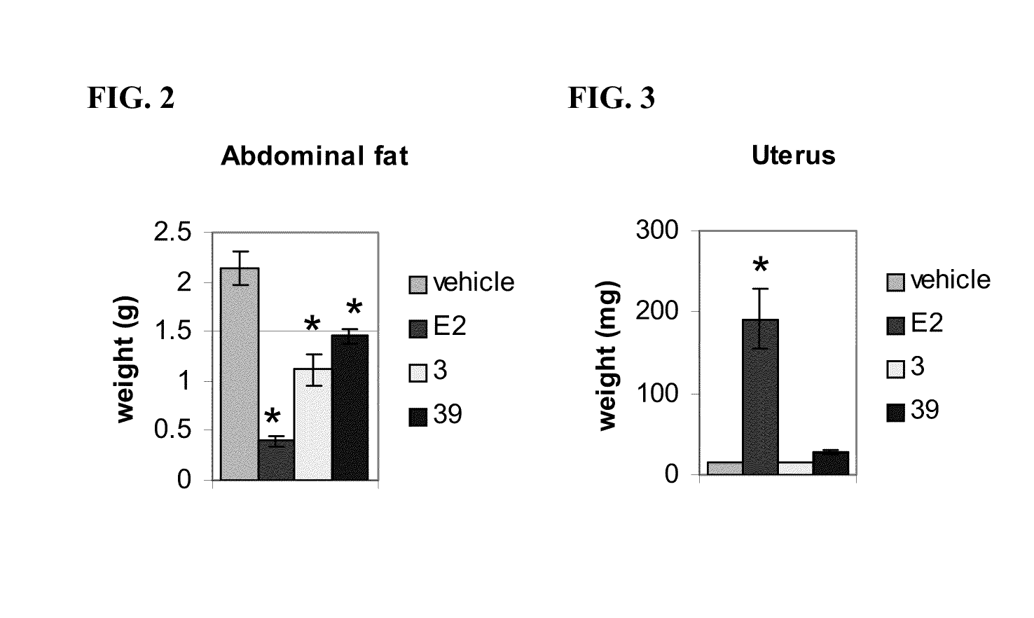

To test the effects of the two PEs (3 and 39) in animals, 10 week old ovariectomized C57/B16 female mice were placed on a high fat diet (HFD) for 50 days to increase body weight and abdominal fat. After gaining weight, the mice were treated orally for 6 weeks with the two PEs separately while being kept on HFD. The vehicle-treated (placebo) control mice continued to gain weight (9% weight increase) even after treatment. The E2-treated mice, which served as positive controls lost 20.5% of their weight. The body weight of both PE-treated groups was significantly reduced to levels similar to the E2 treated mice. Weight gain/loss by each group of animals is shown in FIG. 1.

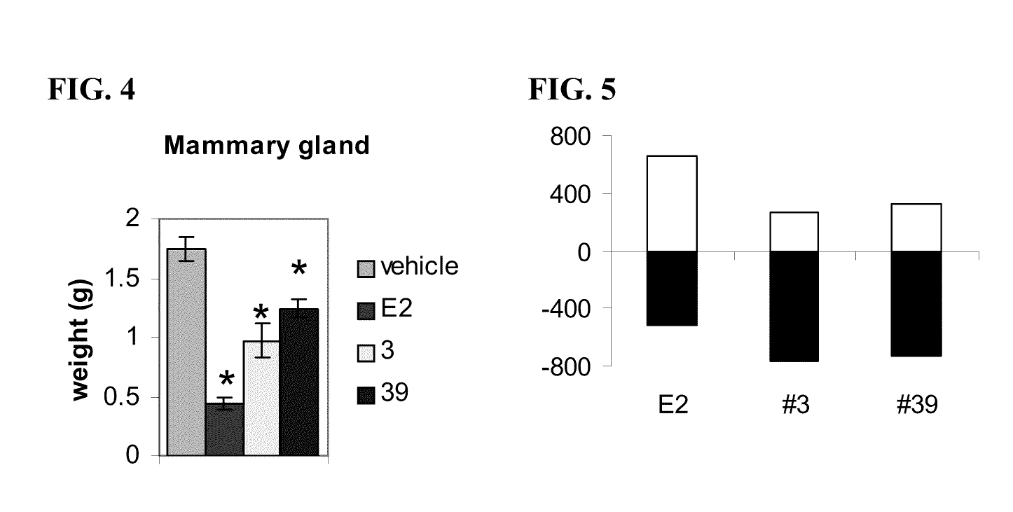

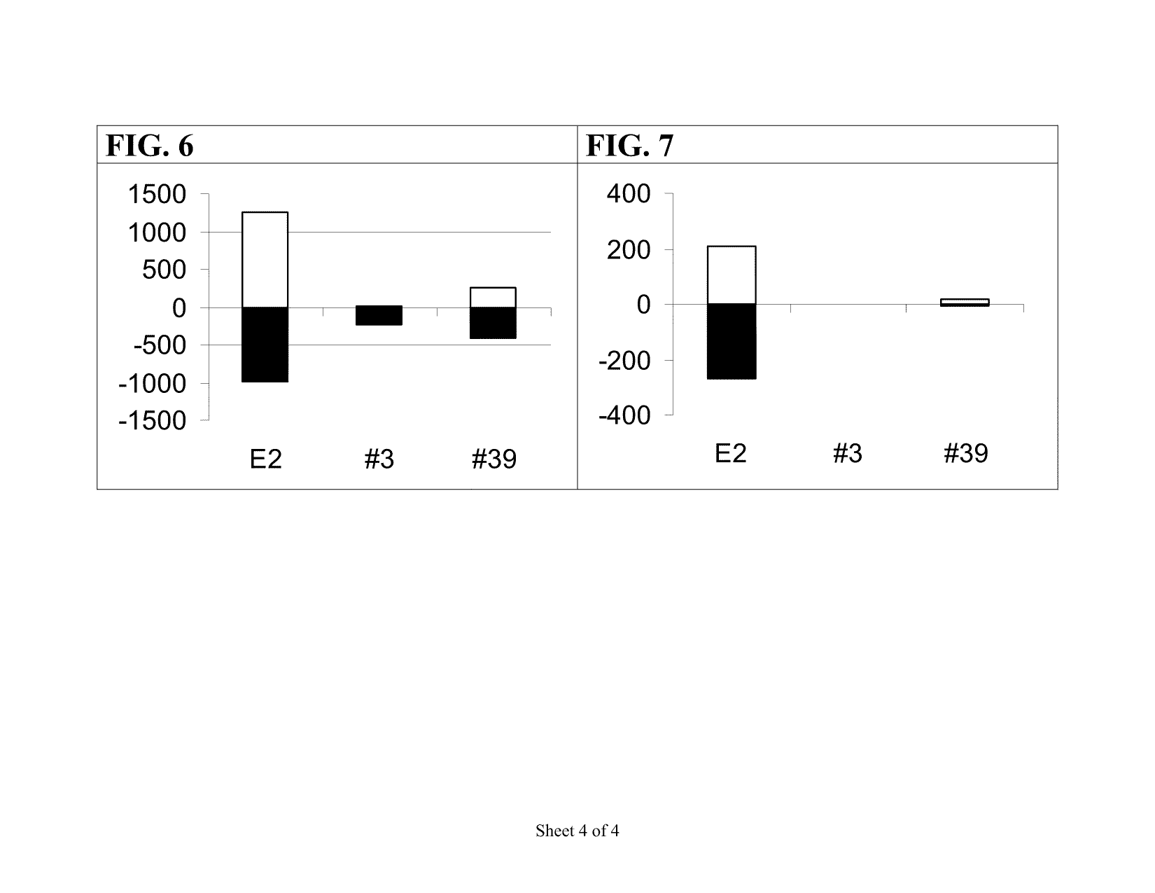

The abdominal fat weight dropped by 48% or 32% in the PE-treated groups (3, 39, respectively) and 80% in E2-treated mice compared to control. The growth of mammary gland (MG) and uteri was assessed by measuring MG and uterine weights and by analyzing MG morphology in whole-mounts. The mean weights, in grams, of the uteri and MG obtained from the mice as shown in FIGS. 3 and 4, respectively. The MGs of control mice weighed 4 times more than those from the E2-treated mice, even though histology revealed that the control glands had less ductal structures. FIG. 4. The MGs of PE-treated mice have a phenotype similar to control with few ductal structures, demonstrating the absence of epithelial cell proliferation. Finally, the uterine weights in both PE-treated groups were similar to the control mice, whereas the E2-treated uteri were 18 times heavier than controls. See FIG. 3. These studies show that these plant-derived ERαagonists have favorable tissue-specific effects because they reversed fat accumulation without causing proliferative effects on the MG and uterus, in contrast to the estrogens used currently in HRT.

These results demonstrate the potential of the tested PEs (3, 39) to reduce abdominal accumulation of body fat in per-menopausal, postmenopausal and menopausal women. These results also demonstrate the potential of the tested PEs to reduce weight in the same demographic group. These results imply the potential for inducing additional concomitant effects on the same demographic group, including reducing occurrence or severity of metabolic syndrome, cardiovascular disease, delaying onset and/or severity of type 2 diabetes, etc.

### Example 2

### Reduction of Body Fat Accumulation or Induction of Weight Loss in Menopausal Women

Menopausal (perimenopausal or postmenopausal) women are treated daily for 1 month to 3 months with either a control solution consisting of a pharmaceutically acceptable vehicle (e.g. water, sugar water or flavored water) or 1 mg, 10 mg, 100 mg or 1000 mg of Radix Glycyrrhiza extract in vehicle, Radix Pueraria extract in vehicle, an extract of a combination of Radix Glycyrrhiza and Radix Pueraria in vehicle. The vehicle-treated women act as the control arm. The women treated with herbal extracts are weighed, and their heights and other bodily dimensions (e.g. waist, hip and bust measurements) are determined at the beginning of the study and at 1, 2 or 4 week intervals after beginning the study. Body mass indices and body fat estimates and fat distribution are obtained either from the weight and body dimension measurements, from skin caliper measurements, from underwater weighing, or two or more thereof. The endpoints of the study are body weight loss, body fat distribution, abdominal circumference decrease, body fat decrease and body mass index decrease.

### CONCLUSION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of reducing fat accumulation, inducing weight loss, or both, comprising administering to a subject an estrogen receptor alpha (ERα) agonizing amount of a plant extract comprising one or more members selected from the group consisting of extracts of Radix Anamarrhena, Radix Glycyrrhiza, Radix Pueraria, Herba Epimedii, and combinations thereof.

2. The method of claim 1, wherein the plant extract comprises two or more members selected from the group consisting of extracts of Radix Anamarrhena, Radix Glycyrrhiza, Radix Pueraria, and Herba Epimedii.

3. The method of claim 1, wherein the plant extract comprises three or more members selected from the group consisting of extracts of Radix Anamarrhena, Radix Glycyrrhiza, Radix Pueraria, and Herba Epimedii.

4. The method of claim 1, wherein the plant extract comprises extracts of Radix Anamarrhena, Radix Glycyrrhiza, Radix Pueraria, and Herba Epimedii.

5. The method of claim 1, wherein the plant extract comprises extracts of Radix Glycyrrhiza, Radix Pueraria, and combinations thereof.

6. The method of claim 1, wherein the plant extract comprises a combination of extracts of Radix Glycyrrhiza and Radix Pueraria.

7. The method of claim 1, wherein the plant extract comprises an ethanol:water extract of Radix Glycyrrhiza or Herba Epimedii.

8. The method of claim 1, wherein the plant extract comprises an ethyl acetate partition of an extract of Radix Anamarrhena, Radix Glycyrrhiza, Radix Pueraria, or Herba Epimedii.

9. The method of claim 1, wherein the plant extract comprises an aqueous extract of Herba Epimedii.

10. The method of claim 1, wherein the plant extract comprises a mixture of two or more of: an ethanol:water extract of Radix Glycyrrhiza or Herba Epimedii, an ethyl acetate partition of an extract of Radix Anamarrhena, Radix Glycyrrhiza, Radix Pueraria, or Herba Epimedii, or an aqueous extract of Herba Epimedii.

11. The method of claim 1, wherein the method produces a reduction in abdominal fat.

12. A composition for reducing fat accumulation, inducing weight loss, or both, comprising an estrogen receptor alpha (ERα) agonizing amount of a plant extract comprising one or more members selected from the group consisting of extracts of Radix Anamarrhena, Radix Glycyrrhiza, Radix Pueraria, Herba Epimedii, and combinations thereof.

13. The composition of claim 12, wherein the plant extract comprises two or more members selected from the group consisting of extracts of Radix Anamarrhena, Radix Glycyrrhiza, Radix Pueraria, and Herba Epimedii.

14. The composition of claim 12, wherein the plant extract comprises three or more members selected from the group consisting of extracts of Radix Anamarrhena, Radix Glycyrrhiza, Radix Pueraria, and Herba Epimedii.

15. The composition of claim 12, wherein the plant extract comprises extracts of Radix Anamarrhena, Radix Glycyrrhiza, Radix Pueraria, and Herba Epimedii.

16. The composition of claim 12, wherein the plant extract comprises extracts of Radix Glycyrrhiza, Radix Pueraria, and combinations thereof.

17. The composition of claim 12, wherein the plant extract comprises a combination of extracts of Radix Glycyrrhiza and Radix Pueraria.

18. The composition of claim 12, wherein the plant extract comprises an ethanol:water extract of Radix Glycyrrhiza or Herba Epimedii.

19. The composition of claim 12, wherein the plant extract comprises an ethyl acetate partition of an extract of Radix Anamarrhena, Radix Glycyrrhiza, Radix Pueraria, or Herba Epimedii.

20. The composition of claim 12, wherein the plant extract comprises an aqueous extract of Herba Epimedii.

21. The composition of claim 12, wherein the plant extract comprises a mixture of two or more of: an ethanol:water extract of Radix Glycyrrhiza or Herba Epimedii, an ethyl acetate partition of an extract of Radix Anamarrhena, Radix Glycyrrhiza, Radix Pueraria, or Herba Epimedii, or an aqueous extract of Herba Epimedii.

22. A method of reducing fat accumulation, inducing weight loss, or both, comprising administering to a menopausal, perimenopausal or postmenopausal subject an estrogen receptor alpha (ERα) agonizing amount of a plant extract comprising one or more members selected from the group consisting of extracts of Radix Anamarrhena, Radix Glycyrrhiza, Radix Pueraria, Herba Epimedii, and combinations thereof.

23. The method of claim 22, wherein the plant extract comprises two or more members selected from the group consisting of extracts of Radix Anamarrhena, Radix Glycyrrhiza, Radix Pueraria, and Herba Epimedii.

24. The method of claim 22, wherein the plant extract comprises three or more members selected from the group consisting of extracts of Radix Anamarrhena, Radix Glycyrrhiza, Radix Pueraria, and Herba Epimedii.

25. The method of claim 22, wherein the plant extract comprises extracts of Radix Anamarrhena, Radix Glycyrrhiza, Radix Pueraria, and Herba Epimedii.

26. The method of claim 22, wherein the method produces a reduction in abdominal fat accumulation.

27. The method of claim 22, wherein the plant extract comprises extracts of Radix Glycyrrhiza, Radix Pueraria, and combinations thereof.

28. The method of claim 22, wherein the plant extract comprises a combination of extracts of Radix Glycyrrhiza and Radix Pueraria.

29. The method of claim 22, wherein the plant extract comprises an ethanol:water extract of Radix Glycyrrhiza or Herba Epimedii.

30. The method of claim 22, wherein the plant extract comprises an ethyl acetate partition of an extract of Radix Anamarrhena, Radix Glycyrrhiza, Radix Pueraria, or Herba Epimedii.

31. The method of claim 22, wherein the plant extract comprises an aqueous extract of Herba Epimedii.

32. The method of claim 22, wherein the plant extract comprises a mixture of two or more of: an ethanol:water extract of Radix Glycyrrhiza or Herba Epimedii, an ethyl acetate partition of an extract of Radix Anamarrhena, Radix Glycyrrhiza, Radix Pueraria, or Herba Epimedii, or an aqueous extract of Herba Epimedii.

33. A method of selectively agonizing Estrogen Receptor alpha (ERα) in adipose tissue, comprising administering an effective amount of a plant extract comprising one or more members selected from the group consisting of extracts of Radix Anamarrhena, Radix Glycyrrhiza, Radix Pueraria, Herba Epimedii, and combinations thereof.

34. The method of claim 33, wherein the plant extract comprises two or more members selected from the group consisting of extracts of Radix Anamarrhena, Radix Glycyrrhiza, Radix Pueraria, and Herba Epimedii.

35. The method of claim 33, wherein the plant extract comprises three or more members selected from the group consisting of extracts of Radix Anamarrhena, Radix Glycyrrhiza, Radix Pueraria, and Herba Epimedii.

36. The method of claim 33, wherein the plant extract comprises extracts of Radix Anamarrhena, Radix Glycyrrhiza, Radix Pueraria, and Herba Epimedii.

37. The method of claim 33, wherein the method produces a reduction in abdominal fat accumulation.

38. The method of claim 33, wherein the plant extract comprises extracts of Radix Glycyrrhiza, Radix Pueraria, and combinations thereof.

39. The method of claim 33, wherein the plant extract comprises a combination of extracts of Radix Glycyrrhiza and Radix Pueraria.

40. The method of claim 33, wherein the plant extract comprises an ethanol:water extract of Radix Glycyrrhiza or Herba Epimedii.

41. The method of claim 33, wherein the plant extract comprises an ethyl acetate partition of an extract of Radix Anamarrhena, Radix Glycyrrhiza, Radix Pueraria, or Herba Epimedii.

42. The method of claim 33, wherein the plant extract comprises an aqueous extract of Herba Epimedii.

43. The method of claim 33, wherein the plant extract comprises a mixture of two or more of: an ethanol:water extract of Radix Glycyrrhiza or Herba Epimedii, an ethyl acetate partition of an extract of Radix Anamarrhena, Radix Glycyrrhiza, Radix Pueraria, or Herba Epimedii, or an aqueous extract of Herba Epimedii.

44. A method of reducing fat accumulation, inducing weight loss, or both, comprising administering to a subject in need of such reduction in fat accumulation, induction of weight loss, or both, an effective amount of a plant extract comprising one or more members selected from the group consisting of extracts of Radix Anamarrhena, Radix Glycyrrhiza, Radix Pueraria, Herba Epimedii, and combinations thereof.

45. The method of claim 44, wherein the plant extract comprises two or more members selected from the group consisting of extracts of Radix Anamarrhena, Radix Glycyrrhiza, Radix Pueraria, and Herba Epimedii.

46. The method of claim 44, wherein the plant extract comprises three or more members selected from the group consisting of extracts of Radix Anamarrhena, Radix Glycyrrhiza, Radix Pueraria, and Herba Epimedii.

47. The method of claim 44, wherein the plant extract comprises extracts of Radix Anamarrhena, Radix Glycyrrhiza, Radix Pueraria, and Herba Epimedii.

48. The method of claim 44, wherein the method produces a reduction in abdominal fat accumulation.

49. The method of claim 44, wherein the plant extract comprises extracts of Radix Glycyrrhiza, Radix Pueraria, and combinations thereof.

50. The method of claim 44, wherein the plant extract comprises a combination of extracts of Radix Glycyrrhiza and Radix Pueraria.

51. The method of claim 44, wherein the plant extract comprises an ethanol:water extract of Radix Glycyrrhiza or Herba Epimedii.

52. The method of claim 44, wherein the plant extract comprises an ethyl acetate partition of an extract of Radix Anamarrhena, Radix Glycyrrhiza, Radix Pueraria, or Herba Epimedii.

53. The method of claim 44, wherein the plant extract comprises an aqueous extract of Herba Epimedii.

54. The method of claim 44, wherein the plant extract comprises a mixture of two or more of: an ethanol:water extract of Radix Glycyrrhiza or Herba Epimedii, an ethyl acetate partition of an extract of Radix Anamarrhena, Radix Glycyrrhiza, Radix Pueraria, or Herba Epimedii, or an aqueous extract of Herba Epimedii.
